(19) United States
(12) Patent Application Publication
(10) Pub. No.: US 2013/0204187 A1
(43) Pub. Date: Aug. 8, 2013

(54) METHOD AND SYSTEM FOR RETAINING A CARTRIDGE IN A HOLDER

(71) Applicants: Richard James Vincent Avery, Chipping Campden (GB); Joseph Butler, Rugby (GB)

(72) Inventors: Richard James Vincent Avery, Chipping Campden (GB); Joseph Butler, Rugby (GB)

(73) Assignee: SANOFI-AVENTIS DEUTSCHLAND GMBH, Frankfurt am Main (DE)

(21) Appl. No.: 13/810,505

(22) PCT Filed: Aug. 5, 2011
(86) PCT No.: PCT/EP2011/063502

§ 371 Date: Apr. 26, 2013

Related U.S. Application Data
(60) Provisional application No. 61/371,238, filed on Aug. 6, 2010.

(30) Foreign Application Priority Data
Sep. 2, 2010 (EP) 10175001.6

(51) Int. Cl. A61M 5/50

(52) U.S. Cl. CPC A61M 5/5086
USPC 604/111; 604/187

(57) ABSTRACT

A method and system for retaining a drug cartridge in a cartridge holder. As an example, a retaining member for retaining a drug cartridge in a cartridge holder includes at least one connection feature for connecting to the drug cartridge. The at least one connection feature is on an outer wall of the retaining member. The retaining member further includes at least one retention feature, and this at least one retention feature extends from an inner wall of the retaining member. When the retaining member is connected to the cartridge holder, the retaining member retains the drug cartridge in the cartridge holder.

### CROSS REFERENCE TO RELATED APPLICATIONS

The present application is a U.S. National Phase Application pursuant to 35 U.S.C. §371 of International Application No. PCT/EP2011/063502 filed Aug. 5, 2011, which claims priority to U.S. Provisional Patent Application No. 61/371,238 filed Aug. 6, 2010, and European Patent Application No. 10175001.6, filed Sep. 2, 2010. The entire disclosure contents of these applications are herewith incorporated by reference into the present application.

### FIELD OF INVENTION

Specific embodiments of this disclosure relate to reservoirs, particularly reservoirs containing a medicament. More particularly, the present disclosure is generally directed to retaining a reservoir in a cartridge holder. The cartridge holder may then be coded for use with a drug delivery device so as to prevent unwanted reservoir cross use. As just one example, such medicament reservoirs may comprise an ampoule, a cartridge, a vial, or a pouch, and may be used with a medical delivery device. Exemplary medical delivery devices include, but are not limited to syringes, pen-type injection syringes, pumps, inhalers, or other similar injection or infusing devices that require at least one reservoir containing at least one medicament.

### BACKGROUND

Medicament reservoirs such as ampoules, cartridges, or vials are generally known. Such reservoirs are especially used for medicaments that may be self administered by a patient. For example, with respect to insulin, a patient suffering from diabetes may require a certain amount of insulin to either be injected via a pen-type injection syringe or infused via a pump. With respect to certain known reusable pen-type drug delivery devices, a patient may load a cartridge containing the insulin into a proximal end of a cartridge holder. After the cartridge has been correctly loaded, the user may then be called upon to select a dose of medicament. Multiple doses may be dosed from the cartridge. Where the drug delivery device comprises a reusable device, once the cartridge is empty, the cartridge holder may be disconnected from the drug delivery device and the empty cartridge is removed and replaced with a new cartridge. Most suppliers of such cartridges recommend the user to dispose of the empty cartridges properly. Where the drug delivery device comprises a disposable device, once the cartridge is empty, the user is recommended to dispose of the entire device.

Such known self administration systems requiring the removal and reloading of empty cartridges have certain limitations. For example, in certain generally known systems, a user simply may load a new cartridge into the delivery system without the drug delivery device or without the cartridge having any mechanism of preventing cross use of an incorrect cartridge. That is, the drug delivery device does not have a mechanism for determining whether the medicament contained in the cartridge is indeed the correct type of medicament to be administered by the patient. Alternatively, certain known drug delivery devices do not present a mechanism for determining whether the correct type of medicament within the cartridge should be used with that particular drug delivery system. This potential problem could be exacerbated given that certain elderly patients, such as those suffering from diabetes, may have limited manual dexterity. Identifying an incorrect medicament is quite important, since the administration of a potentially incorrect dose of a medicament such as a short-acting insulin in lieu of a long-acting insulin could result in injury or even death.

Some drug delivery devices or systems may use a color coding scheme to assist a user or care giver in selecting the correct cartridge to be used with a drug delivery device. However, such color coding schemes pose challenges to certain users, especially those users suffering from poor eyesight or color blindness: a situation that can be quite prevalent in patients suffering from diabetes.

Another concern that may arise with such disposable cartridges is that these cartridges are manufactured in essentially standard sizes and manufactured to comply with certain recognized local and international standards. Consequently, such cartridges are typically supplied in standard sized cartridges (e.g. 3 ml cartridges). Therefore, there may be a variety of cartridges supplied by a number of different suppliers and containing a different medicament but they may fit a single drug delivery device. As just one example, a first cartridge containing a first medicament from a first supplier may fit a medical delivery device provided by a second supplier. As such, a user might be able to load and then dispense an incorrect medicament (such as a rapid or basal type of insulin) into a drug delivery device without being aware that the medical delivery device was perhaps neither designed nor intended to be used with such a cartridge.

As such, there is a growing desire from users, health care providers, care givers, regulatory entities, and medical device suppliers to reduce the potential risk of a user loading an incorrect drug type into a drug delivery device. There is also, therefore, a desire to reduce the risk of dispensing an incorrect medicament (or the wrong concentration of the medicament) from such a drug delivery device.

There is, therefore, a general need to physically dedicate or mechanically code a cartridge to its drug type and design an injection device that only accepts or works with the dedication or coded features provided on or with the cartridge so as to prevent unwanted cartridge cross use. Similarly, there is also a general need for a dedicated cartridge that allows the medical delivery device to be used with only an authorized cartridge containing a specific medicament while also preventing undesired cartridge cross use.

There is also a general need to provide a dedicated cartridge that is difficult to tamper with so that the cartridge may not be compromised in that the cartridge can be used with an unauthorized drug or drug delivery device. Because such cartridges may be difficult to tamper with, they may also reduce the risk of counterfeiting: i.e. making it more difficult for counterfeiters to provide unregulated counterfeit medicament carrying products.

These and other advantages and features will become evident from the following more detailed description of the invention.

One problem to be solved by the present invention is to provide a cartridge assembly where the safety of the user is increased.

### SUMMARY

One aspect relates to a cartridge assembly for connection to a drug delivery device. The cartridge assembly may comprise a cartridge holder. A drug cartridge may be held in the cartridge holder. The cartridge assembly may comprise a retaining member. The retaining member may be a retaining ring. Alternatively, the retaining member may be a split ring, which is described later on in more detail. Alternatively, the retaining member may be a clip, which is described later on in more detail. Alternatively, the retaining member may comprise a resin or an adhesive, which is described later on in more detail. The retaining member may be connected to the cartridge holder so that the retaining member may permanently or releasably retain the drug cartridge in the cartridge holder.

One aspect relates to a retaining ring. The retaining ring may be configured for permanently or releasably retaining a drug cartridge in a cartridge holder. The retaining ring may comprise at least one connection feature, preferably two or more connection features. The connection feature may be connected for permanently or releasably connecting the retaining ring to the drug cartridge and/or to the cartridge holder. The at least one connection feature may be on an outer wall of the retaining ring. The cartridge holder and/or the retaining ring may further include at least one retention feature, preferably two or more retention features. This at least one retention feature may extend from an inner wall of the retaining ring. When the retaining ring is releasably or permanently connected to the cartridge holder, the retaining ring may retain the drug cartridge in the cartridge holder.

According to an embodiment, the retaining ring is connected to a proximal end of the cartridge holder. Alternatively, the retaining ring may be connected to a distal end of the cartridge holder. In the latter case, the retention features may permanently or releasably retain a ferrule of the drug cartridge.

According to an embodiment, the at least one connection feature is a threaded connection feature. Additionally or alternatively, the at least one connection feature may comprise at least one protrusion. The protrusion may be capable of snapping into an indentation in the proximal end of the cartridge holder.

According to an embodiment, the retaining ring comprises at least one ratchet feature. The ratchet feature may be located on the outer wall of the retaining ring.

According to an embodiment, the at least one retention feature is a protrusion. The protrusion may protrude along an entire circumference of the inner wall of the cartridge holder, for example. Additionally or alternatively, the at least one retention feature may comprise at least one of, preferably a plurality of tabs. The tabs may extend from the inner wall of the retaining ring.

According to an embodiment, a fastening mechanism may be provided on the retaining ring. The fastening mechanism may be for permanently or releasably attaching to a drug delivery device. The fastening means may be located on the outer wall of the retaining ring.

According to an embodiment, the ring is a generally circular shape.

According to an embodiment, the retaining ring is permanently or releasably connected to the cartridge holder. At least one of the retaining ring and cartridge holder may be mechanically coded to a given drug delivery device.

According to an embodiment, the retaining ring is flexible such that the ring biases the cartridge, in particular toward a distal end of the cartridge holder.

A further aspect relates to a cartridge assembly. The cartridge assembly may be for connection to a drug delivery device. The cartridge assembly may comprise a cartridge holder. A drug cartridge may be held in the cartridge holder. A split ring, e.g. the previously described retaining ring, may be disposed between a portion of the cartridge holder and a portion of the drug cartridge. The split ring may permanently or releasably retain the cartridge in the cartridge holder.

According to an embodiment, at least one of the split ring and holder is flexible. An angle of at least one contact face between the split ring and the cartridge holder may allow for assembly of the cartridge assembly. The angle may prevent disassembly of the cartridge assembly.

According to an embodiment, the split ring is disposed between a neck of the cartridge and an inner wall of the cartridge holder.

According to an embodiment, the cartridge holder has a groove. The groove may extend along at least a portion of the circumference of an inner wall of the holder. The groove may permanently or releasably hold the split ring. A diameter of the split ring may be larger than a diameter of the groove. The groove may be located such that the split ring held in the groove will contact a ferrule, preferably a proximal side of the ferrule, of the drug cartridge when the drug cartridge is fully inserted into the holder.

According to an embodiment, the split ring comprises a first semi-circular portion. The first semi-circular portion may be hinged to a second semi-circular portion. The first portion may comprise a first snap feature. The second portion may comprise a second snap feature. The first and second snap features may be snapped together.

According to an embodiment, the split ring is an axially flexible ring. The axially flexible ring may bias the cartridge in a distal direction.

A further aspect relates to a cartridge assembly. The cartridge assembly may be for connection to a drug delivery device. The cartridge assembly may include a cartridge holder. A drug cartridge may be held in the cartridge holder. The cartridge assembly may include at least one of an adhesive and a resin. The at least one of an adhesive or a resin may be disposed between a portion of the cartridge holder and a portion of the drug cartridge. The at least one of an adhesive and a resin may permanently or releasably retain the cartridge in the cartridge holder.

According to an embodiment, a neck of the cartridge holder comprises at least one injection port through which the adhesive may be injected.

According to an embodiment, a neck of the holder comprises a groove extending along at least a portion of the circumference of an inner wall of the holder.

According to an embodiment, the drug cartridge includes a ferrule. The ferrule may have a preferably detachable membrane over the ferrule. The preferably detachable membrane may prevent adhesive from touching a septum in the ferrule.

According to an embodiment, the at least one of an adhesive and a resin is selected from the group consisting of polypropylene, polyurethane, polyester, or epoxy.

According to an embodiment, the cartridge assembly, in particular preferably at least one of the cartridge holder and the cartridge, is mechanically coded to a given drug delivery device.

A further aspect relates to a cartridge holder. The cartridge holder may comprise at least one fastening feature. The fastening feature may be for permanently or releasably connecting to a drug delivery device. The at least one fastening feature may be located on a proximal end of the cartridge holder. The holder may further include at least one retention feature, preferably two or more retention features. When a drug cartridge is held in the cartridge holder, the retention features may prevent the drug cartridge from moving past a proximal end of the cartridge holder.

According to an embodiment, the at least one retention feature is located on a proximal end of the cartridge holder. The at least one retention feature may be a ring-shaped protrusion. The ring-shaped protrusion may be around the proximal end. Alternatively, the at least one retention feature may be located at a distal end of the cartridge holder. Said retention features may prevent movement of a ferrule of the drug cartridge.

According to an embodiment, the at least one retention feature comprises a plurality of protrusions.

According to an embodiment, the cartridge holder comprises a flexible section. The flexible section may facilitate biasing the drug cartridge when the drug cartridge is held in the holder.

According to an embodiment, the cartridge holder is mechanically coded to a given drug delivery device.

According to an embodiment, a body of the cartridge holder is split into a first body portion and a second body portion. Each body portion may comprise connection features. The connection features may be for permanently or releasably connecting to one another.

A further aspect relates to a cartridge assembly. The cartridge assembly may be for connection to a drug delivery device. The cartridge assembly may comprise a cartridge holder. The cartridge holder may comprise a neck portion. A drug cartridge may be held in the cartridge holder. The cartridge assembly may comprise a clip, preferably the previously described retaining ring. The neck portion may comprise at least one hole. The hole may be for receiving the clip. When the clip is inserted into the hole, the clip may permanently or releasably retain the drug cartridge in the cartridge holder.

According to an embodiment, the neck portion comprises a stop feature. The stop feature may stop the clip when the clip is inserted into the hole.

According to an embodiment, the neck portion further comprises at least one surface. The surface may limit axial movement of the clip relative to the cartridge holder.

According to an embodiment, the clip comprises snap features. The sap features may snap around the neck of the cartridge when the clip is inserted into the hole.

According to an embodiment, the holder comprises connection features. The connection features may be on an inner wall of the neck of the holder. The clip may comprise corresponding connection features. The corresponding connection features may fit into the connection features of the holder.

According to an embodiment, a length of the clip is longer than a length of the hole. During insertion of the clip into the hole, the clip may be compressed and may snap into to the connection features.

According to an embodiment, a body of the clip is flush with a circumference of the neck when the clip is fully inserted.

According to a preferred embodiment, a cartridge assembly for connection to a drug delivery device is provided, the cartridge assembly comprising, a cartridge holder, a drug cartridge being held in the cartridge holder and a retaining member. The retaining member is connected to the cartridge holder so that the retaining member retains the drug cartridge in the cartridge holder.

The retaining member may comprise the previously described retaining ring. The retaining member may comprise the previously described split ring. The retaining member may comprise the previously described clip. The retaining member may comprise the previously described adhesive or resin. The retaining member may be configured for retaining the cartridge in the holder.

According to a preferred embodiment, a retaining ring for retaining a drug cartridge in a cartridge holder is provided, the retaining ring comprising at least one connection feature for connecting to the cartridge holder, wherein the at least one connection feature is on an outer wall of the retaining ring. The retaining ring comprises at least one retention feature, wherein the at least one retention feature extends from an inner wall of the retaining ring, and wherein, when the retaining ring is connected to the cartridge holder, the retaining ring retains the drug cartridge in the cartridge holder.

According to a preferred embodiment, a cartridge assembly for connection to a drug delivery device is provided, the cartridge assembly comprising a cartridge holder, a drug cartridge being held in the cartridge holder, a split ring disposed between a portion of the cartridge holder and a portion of the drug cartridge, wherein the split ring retains the cartridge in the cartridge holder.

According to a preferred embodiment, a cartridge assembly for connection to a drug delivery device is provided, the cartridge assembly comprising a cartridge holder, a drug cartridge being held in the cartridge holder, and at least one of an adhesive and a resin disposed between a portion of the cartridge holder and a portion of the drug cartridge, wherein the at least one of an adhesive and a resin retains the cartridge in the cartridge holder.

According to a preferred embodiment, a cartridge assembly for connection to a drug delivery device is provided, the cartridge assembly comprising a cartridge holder and a drug cartridge held in the cartridge holder. The cartridge holder comprises at least one fastening feature for connecting to a drug delivery device and at least one retention feature. When the drug cartridge is held in the cartridge holder, the retention feature prevents the drug cartridge from moving past a proximal end of the cartridge holder.

According to a preferred embodiment, a cartridge holder is provided comprising: at least one fastening feature for connecting to a drug delivery device, wherein the at least one fastening feature is located on a proximal end of the cartridge holder. The cartridge holder comprises at least one retention feature, wherein, when a drug cartridge is held in the cartridge holder, the retention feature prevents the drug cartridge from moving past a proximal end of the cartridge holder.

According to a preferred embodiment, a cartridge assembly for connection to a drug delivery device is provided, the cartridge assembly comprising a cartridge holder comprising a neck portion, a drug cartridge being held in the cartridge holder, and a clip, wherein the neck portion comprises at least one hole for receiving the clip, wherein the clip is inserted into the hole, and wherein the clip retains the drug cartridge in the cartridge holder.

These as well as other advantages of various aspects of the present invention will become apparent to those of ordinary skill in the art by reading the following detailed description, with appropriate reference to the accompanying figures.

The scope of the invention is defined by the content of the claims. The invention is not limited to specific embodiments but comprises any combination of elements of different embodiments. Moreover, the invention comprises any combination of claims and any combination of features disclosed by the claims.

### BRIEF DESCRIPTION OF THE FIGURES

Exemplary embodiments are described herein with reference to the figures, in which:

FIG. 1A illustrates an exemplary pen-type drug delivery device;

FIG. 1B illustrates an exemplary drug cartridge;

FIG. 2A depicts a perspective view of an exemplary cartridge holder that has an exemplary retaining ring attached;

FIG. 2B depicts a perspective cross-sectional view of an exemplary cartridge holder that has an exemplary retaining ring attached;

FIG. 3A depicts a perspective view of an exemplary retaining ring;

FIG. 3B depicts a perspective view of an exemplary cartridge holder that has the exemplary retaining ring of FIG. 3A attached;

FIG. 4A depicts a perspective view of an exemplary cartridge holder and an exemplary retaining ring;

FIG. 4B depicts a cross-sectional view of the exemplary cartridge holder of FIG. 4A attached to the exemplary retaining ring of FIG. 4A;

FIG. 5A depicts a cross-sectional perspective view of an exemplary cartridge holder attached to an exemplary snap-on retaining ring;

FIG. 5B depicts a cross-sectional view of an exemplary snap-on retaining ring;

FIG. 6 depicts a perspective cross-sectional view of exemplary cartridge retained in an exemplary cartridge holder by an exemplary split ring;

FIG. 7 depicts a cross-sectional view of a distal end of an exemplary cartridge retained in an exemplary cartridge holder by an exemplary adhesive;

FIG. 8 depicts a cross-sectional view of a distal end of an exemplary cartridge retained in an exemplary cartridge holder by an exemplary adhesive;

FIG. 9 depicts a perspective view of a proximal end of an exemplary cartridge holder;

FIG. 10 depicts a cross-sectional view of the exemplary cartridge holder of FIG. 9;

FIG. 11A depicts a perspective view of a proximal end of an exemplary cartridge holder;

FIG. 11B depicts a perspective view of the exemplary cartridge holder of FIG. 11A;

FIG. 12 depicts a cross-sectional view of an exemplary cartridge holder;

FIG. 13 depicts a perspective view of an exemplary cartridge holder;

FIG. 14A depicts a perspective view of an exemplary cartridge holder, and exemplary cartridge, and an exemplary external clip for retaining the cartridge in the holder;

FIG. 14B depicts a cross-sectional perspective view of the exemplary cartridge holder of FIG. 14A and a perspective view of the exemplary cartridge an external clip of FIG. 14A;

FIG. 15A depicts a perspective view of an exemplary cartridge holder, an exemplary cartridge, and an exemplary external clip for retaining the cartridge in the holder;

FIG. 15B depicts a cross-sectional perspective view of the exemplary cartridge holder of FIG. 15A and a perspective view of the exemplary cartridge and external clip of FIG. 15A;

FIG. 16A depicts a perspective view of an exemplary cartridge holder, an exemplary cartridge, and an exemplary external clip for retaining the cartridge in the holder;

FIG. 16B depicts a partial perspective view of the exemplary cartridge holder of FIG. 16A and a perspective view of the exemplary cartridge and external clip of FIG. 16A;

FIG. 17A depicts a perspective view of an exemplary cartridge holder, an exemplary cartridge, and an exemplary external clip for retaining the cartridge in the holder;

FIG. 17B depicts a partial perspective view of the exemplary cartridge holder of FIG. 17A and a perspective view of the exemplary cartridge and external clip of FIG. 17A;

FIG. 18A depicts a perspective view of an exemplary cartridge holder, an exemplary cartridge, and an exemplary external clip for retaining the cartridge in the holder;

FIG. 18B depicts a partial perspective view of the exemplary cartridge holder of FIG. 18A and a perspective view of the exemplary cartridge and external clip of FIG. 18A;

and

FIG. 19 is a perspective view of an exemplary drug reservoir that may be retained in accordance with the embodiments of the present disclosure.

### DETAILED DESCRIPTION

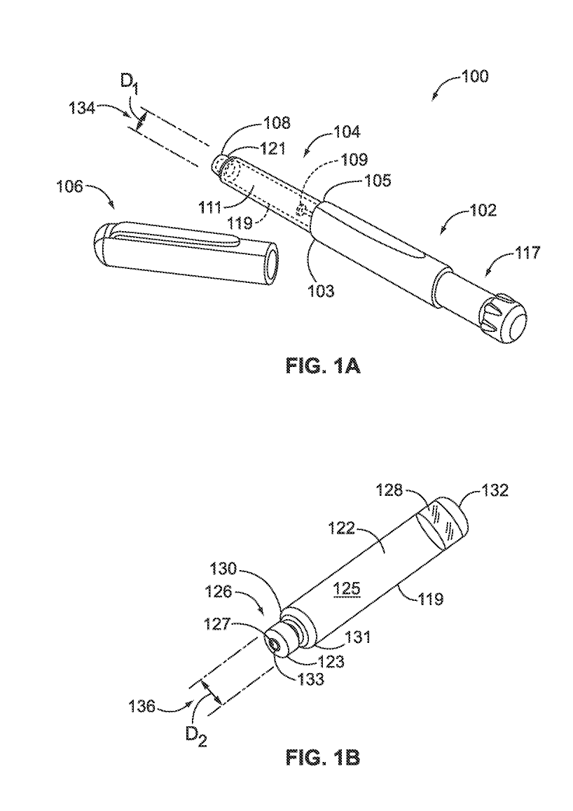

Referring to FIG. 1A, there is shown a drug delivery device 100 in the form of a pen-type syringe. This drug delivery device 100 comprises a dose setting mechanism 102, a cartridge holder 104, and a removable cap 106. A proximal end 105 of the cartridge holder 104 and a distal end 103 of the dose setting mechanism 102 are removably secured together. The pen-type syringe may comprise a re-usable or a disposable pen-type syringe. Where the syringe comprises a re-usable device, the cartridge holder 104 and the dose setting mechanism 102 are removably coupled together. In a disposable device 100, they are permanently coupled together. In FIG. 1, the dose setting mechanism 102 comprises a piston rod 109, such as a threaded piston rod that rotates when a dose is injected.

To inject a previously set dose, a double ended needle assembly is attached to a distal end 108 of the cartridge holder 104. Preferably, the distal end 108 of the holder 104 comprises a thread 121 (or other suitable connecting mechanism such as a snap lock, snap fit, form fit, or bayonet lock mechanism) so that the needle assembly may be removably attached to the distal end 108 of the holder 104. When the drug delivery device 100 is not in use, the removable cap 106 can be releasably retained over the cartridge holder 104.

An inner cartridge cavity 111 defined by the cartridge holder 104 is dimensioned and configured to securely receive and retain a cartridge, such as glass cartridge 119. FIG. 1B illustrates a perspective view of the cartridge 119 that may be used with the drug delivery device 100 illustrated in FIG. 1A. Typically, the cartridge 119 is manufactured of glass and includes a generally tubular barrel 122 extending from a distal end 130 to a proximal end 132.

At the distal end 130, the cartridge 119 includes a smaller diameter neck 126 and this neck 126 projects distally from the shoulder 131 of the barrel 122. Preferably, this smaller diameter neck 126 is provided with a large diameter annular bead 123 and this bead 123 extends circumferentially thereabout at the extreme distal end of the neck 126 and defines an opening 127. A pierceable seal or septum 133 is securely held across the opening 127 by a metallic sleeve or a ferrule (not explicitly shown).

A medicament 125 is pre-filled into the cartridge 119 and is retained within this cartridge 125, in part, by the pierceable seal 133, the ferrule, and a stopper 128.

The term “medicament”, as used herein, preferably means a pharmaceutical formulation containing at least one pharmaceutically active compound,

wherein in one embodiment the pharmaceutically active compound has a molecular weight up to 1500 Da and/or is a peptide, a proteine, a polysaccharide, a vaccine, a DNA, a RNA, an enzyme, an antibody, a hormone or an oligonucleotide, or a mixture of the above-mentioned pharmaceutically active compound,

wherein in a further embodiment the pharmaceutically active compound is useful for the treatment and/or prophylaxis of diabetes mellitus or complications associated with diabetes mellitus such as diabetic retinopathy, thromboembolism disorders such as deep vein or pulmonary thromboembolism, acute coronary syndrome (ACS), angina, myocardial infarction, cancer, macular degeneration, inflammation, hay fever, atherosclerosis and/or rheumatoid arthritis,

wherein in a further embodiment the pharmaceutically active compound comprises at least one peptide for the treatment and/or prophylaxis of diabetes mellitus or complications associated with diabetes mellitus such as diabetic retinopathy,

wherein in a further embodiment the pharmaceutically active compound comprises at least one human insulin or a human insulin analogue or derivative, glucagon-like peptide (GLP-1) or an analogue or derivative thereof, or exedin-3 or exedin-4 or an analogue or derivative of exedin-3 or exedin-4.

Insulin analogues are for example Gly(A21), Arg(B31), Arg(B32) human insulin; Lys(B3), Glu(B29) human insulin; Lys(B28), Pro(B29) human insulin; Asp(B28) human insulin; human insulin, wherein proline in position B28 is replaced by Asp, Lys, Leu, Val or Ala and wherein in position B29 Lys may be replaced by Pro; Ala(B26) human insulin; Des(B28-B30) human insulin; Des(B27) human insulin and Des(B30) human insulin.

Insulin derivates are for example B29-N-myristoyl-des(B30) human insulin; B29-N-palmitoyl-des(B30) human insulin; B29-N-myristoyl human insulin; B29-N-palmitoyl human insulin; B28-N-myristoyl LysB28ProB29 human insulin; B28-N-palmitoyl-LysB28ProB29 human insulin; B30-N-myristoyl-ThrB29LysB30 human insulin; B30-N-palmitoyl-ThrB29LysB30 human insulin; B29-N-(N-palmitoyl-Y-glutamyl)-des(B30) human insulin; B29-N-(N-lithocholyl-Y-glutamyl)-des(B30) human insulin; B29-N-(ω-carboxyheptadecanoyl)-des(B30) human insulin and B29-N-(ω-carboxyhepta [symbol] decanoyl) human insulin.

Exendin-4 for example means Exendin-4(1-39), a peptide of the sequence H His- Gly-Glu-Gly-Thr-Phe-Thr-Ser-Asp-Leu-Ser-Lys-Gln-Met-Glu-Glu-Glu-Ala-Val-Arg-Leu-Phe- Ile-Glu-Trp-Leu-Lys-Asn-Gly-Gly-Pro-Ser-Ser-Gly-Ala-Pro-Pro-Pro-Ser-NH2.

Exendin-4 derivatives are for example selected from the following list of compounds:
H-(Lys)4-des Pro36, des Pro37 Exendin-4(1-39)-NH2,
H-(Lys)5-des Pro36, des Pro37 Exendin-4(1-39)-NH2,
des Pro36 [Asp28] Exendin-4(1-39),
des Pro36 [IsoAsp28] Exendin-4(1-39),
des Pro36 [Met(O)14, Asp28] Exendin-4(1-39),
des Pro36 [Met(O)14, IsoAsp28] Exendin-4(1-39),
des Pro36 [Trp(O2)25, Asp28] Exendin-4(1-39),
des Pro36 [Trp(O2)25, IsoAsp28] Exendin-4(1-39),
des Pro36 [Met(O)14 Trp(O2)25, Asp28] Exendin-4(1-39),
des Pro36 [Met(O)14 Trp(O2)25, IsoAsp28] Exendin-4(1-39); or
des Pro36 [Asp28] Exendin-4(1-39),
des Pro36 [IsoAsp28] Exendin-4(1-39),
des Pro36 [Met(O)14, Asp28] Exendin-4(1-39),
des Pro36 [Met(O)14, IsoAsp28] Exendin-4(1-39),
des Pro36 [Trp(O2)25, Asp28] Exendin-4(1-39),
des Pro36 [Trp(O2)25, IsoAsp28] Exendin-4(1-39),
des Pro36 [Met(O)14 Trp(O2)25, Asp28] Exendin-4(1-39),
des Pro36 [Met(O)14 Trp(O2)25, IsoAsp28] Exendin-4(1-39),
   wherein the group -Lys6-NH2 may be bound to the C-terminus of the Exendin-4 derivative; or an Exendin-4 derivative of the sequence
H-(Lys)6-des Pro36 [Asp28] Exendin-4(1-39)-Lys6-NH2,
des Asp28 Pro36, Pro37, Pro38Exendin-4(1-39)-NH2,
H-(Lys)6-des Pro36, Pro38 [Asp28] Exendin-4(1-39)-NH2,
H-Asn-(Glu)5des Pro36, Pro37, Pro38 [Asp28] Exendin-4(1-39)-NH2,
des Pro36, Pro37, Pro38 [Asp28] Exendin-4(1-39)-(Lys)6-NH2,
H-(Lys)6-des Pro36, Pro37, Pro38 [Asp28] Exendin-4(1-39)-(Lys)6-NH2,
H-Asn-(Glu)5-des Pro36, Pro37, Pro38 [Asp28] Exendin-4(1-39)-(Lys)6-NH2,
H-(Lys)6-des Pro36 [Trp(O2)25, Asp28] Exendin-4(1-39)-Lys6-NH2,
H-des Asp28 Pro36, Pro37, Pro38 [Trp(O2)25] Exendin-4(1-39)-NH2,
H-(Lys)6-des Pro36, Pro37, Pro38 [Trp(O2)25, Asp28] Exendin-4(1-39)-NH2,
H-Asn-(Glu)5-des Pro36, Pro37, Pro38 [Trp(O2)25, Asp28] Exendin-4(1-39)-NH2,
des Pro36, Pro37, Pro38 [Trp(O2)25, Asp28] Exendin-4(1-39)-(Lys)6-NH2,
H-(Lys)6-des Pro36, Pro37, Pro38 [Trp(O2)25, Asp28] Exendin-4(1-39)-(Lys)6-NH2,
H-Asn-(Glu)5-des Pro36, Pro37, Pro38 [Trp(O2)25, Asp28] Exendin-4(1-39)-(Lys)6-NH2,
H-(Lys)6-des Pro36 [Met(O)14, Asp28] Exendin-4(1-39)-Lys6-NH2,
des Met(O)14 Asp28 Pro36, Pro37, Pro38 Exendin-4(1-39)-NH2,
H-(Lys)6-desPro36, Pro37, Pro38 [Met(O)14, Asp28] Exendin-4(1-39)-NH2,
H-Asn-(Glu)5-des Pro36, Pro37, Pro38 [Met(O)14, Asp28] Exendin-4(1-39)-NH2,
des Pro36, Pro37, Pro38 [Met(O)14, Asp28] Exendin-4(1-39)-(Lys)6-NH2,
H-(Lys)6-des Pro36, Pro37, Pro38 [Met(O)14, Asp28] Exendin-4(1-39)-(Lys)6-NH2,
H-Asn-(Glu)5 des Pro36, Pro37, Pro38 [Met(O)14, Asp28] Exendin-4(1-39)-(Lys)6-NH2,
H-Lys6-des Pro36 [Met(O)14, Trp(O2)25, Asp28] Exendin-4(1-39)-Lys6-NH2,
H-des Asp28 Pro36, Pro37, Pro38 [Met(O)14, Trp(O2)25] Exendin-4(1-39)-NH2,
H-(Lys)6-des Pro36, Pro37, Pro38 [Met(O)14, Asp28] Exendin-4(1-39)-NH2,
H-Asn-(Glu)5-des Pro36, Pro37, Pro38 [Met(O)14, Trp(O2)25, Asp28] Exendin-4(1-39)-NH2,
des Pro36, Pro37, Pro38 [Met(O)14, Trp(O2)25, Asp28] Exendin-4(1-39)-(Lys)6-NH2,
H-(Lys)6-des Pro36, Pro37, Pro38 [Met(O)14, Trp(O2)25, Asp28] Exendin-4(S1-39)-(Lys)6-NH2,
H-Asn-(Glu)5-des Pro36, Pro37, Pro38 [Met(O)14, Trp(O2)25, Asp28] Exendin-4(1-39)-(Lys)6-NH2;

or a pharmaceutically acceptable salt or solvate of any one of the afore-mentioned Exedin-4 derivative.

Hormones are for example hypophysis hormones or hypothalamus hormones or regulatory active peptides and their antagonists as listed in Rote Liste, ed. 2008, Chapter 50, such as Gonadotropine (Follitropin, Lutropin, Choriongonadotropin, Menotropin), Somatropine (Somatropin), Desmopressin, Terlipressin, Gonadorelin, Triptorelin, Leuprorelin, Buserelin, Nafarelin, Goserelin.

A polysaccharide is for example a glucosaminoglycane, a hyaluronic acid, a heparin, a low molecular weight heparin or an ultra low molecular weight heparin or a derivative thereof, or a sulphated, e.g. a poly-sulphated form of the above-mentioned polysaccharides, and/or a pharmaceutically acceptable salt thereof. An example of a pharmaceutically acceptable salt of a poly-sulphated low molecular weight heparin is enoxaparin sodium.

Pharmaceutically acceptable salts are for example acid addition salts and basic salts. Acid addition salts are e.g. HCl or HBr salts. Basic salts are e.g. salts having a cation selected from alkali or alkaline, e.g. Na+, or K+, or Ca2+, or an ammonium ion N+(R1)(R2)(R3)(R4), wherein R1 to R4 independently of each other mean: hydrogen, an optionally substituted C1 C6-alkyl group, an optionally substituted C2-C6-alkenyl group, an optionally substituted C6-C10-aryl group, or an optionally substituted C6-C10-heteroaryl group. Further examples of pharmaceutically acceptable salts are described in “Remington's Pharmaceutical Sciences” 17. ed. Alfonso R. Gennaro (Ed.), Mark Publishing Company, Easton, Pa., U.S.A., 1985 and in Encyclopedia of Pharmaceutical Technology.

Pharmaceutically acceptable solvates are for example hydrates.

The stopper 128 is in sliding fluid-tight engagement with the inner tubular wall of the barrel 122. Axially directed forces acting upon the stopper 128 during dose injection or dose administration urges the medication 125 from the cartridge 119 though the double ended needle mounted onto the distal end 130 of the cartridge holder 104 and into the injection site. Such axially directed forces may be provided by the piston rod 109 working in unison with the dose setting mechanism 102.

A portion of the cartridge holder 104 defining the cartridge holder cavity 111 is of substantially uniform diameter represented in FIG. 1 by D1 134. This diameter D1 134 is preferably slightly greater than the diameter D2 136 of the cartridge 119. The interior of the cartridge holder 104 includes an inwardly-extending annular portion or stop that is dimensioned to prevent the cartridge 119 from moving within the cartridge holder 104. In this manner, when the cartridge 119 is loaded into the cavity 111 of the cartridge holder 104 and the cartridge holder 104 is then connected to the dose setting mechanism 102, the cartridge assembly 119 will be held within the cartridge cavity 111.

A number of doses of the medicament 125 may be dispensed from the cartridge 119. Preferably, the cartridge 119 contains a type of medicament 125 that must be administered often, such as one or more times a day. One such medicament 125 is insulin.

The dose setting mechanism 102 comprises a dose setter 117 at the proximal end of the dose setting mechanism 102. In one preferred arrangement, the dose setter 117 is rotated to set a dose. To administer this set dose, the user attaches the needle assembly comprising the double ended needle on the distal end 108 of the cartridge holder 104. In this manner, the needle assembly pierces the seal 133 of the cartridge 119 and is, therefore, in liquid communication with the medicament 25. The user pushes on the dose setter 117 to inject the set dose. The same dose setting and dose administration procedure is followed until the medicament 125 in the cartridge 119 is expended and then, a new cartridge must be loaded in the device 100.

In accordance with embodiments of the present disclosure, a cartridge may be non-detachably (i.e. permanently) retained in a cartridge holder. The cartridge may be retained within a cartridge holder in various ways. Beneficially, a holder that contains a non-detachable cartridge can be mechanically coded to a specific drug delivery device (or specific drug delivery devices), ensuring that a drug can only be used with the correct device (or devices). In some embodiments, a cartridge can be fixed in the holder or biased against the holder, thereby removing the need for a cartridge biasing mechanism within the drug delivery device. If the cartridge is free to float axially in the cartridge holder, then the delivered dose might not be constant for a given advancement of the plunger, due to compliance of the stopper or bung and the septum. By biasing the cartridge in the distal direction, movement of the cartridge can be reduced, and thus the dose accuracy improved. This arrangement also fixes the position of the cartridge relative to the cartridge holder, and hence relative to the delivery device, so that the plunger mechanism can detect the location of the last dose more accurately. In alternative embodiments, a cartridge may be detachably retained in a cartridge holder.

In a first embodiment, a retaining ring may be attached to a cartridge holder in order to securely retain a cartridge in the cartridge holder. The retaining ring preferably includes at least one retention feature disposed on the ring. For example, the retention feature may be an inwardly directed radial protrusion on the retaining ring. This protrusion may seat against the open end of an inserted cartridge and prevent removal of the cartridge from the holder.

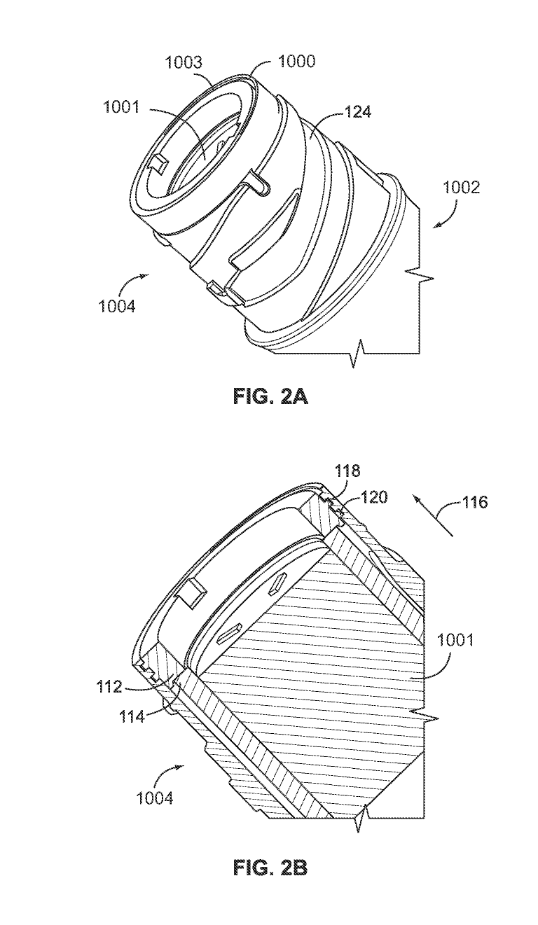

A retaining ring in accordance with this first embodiment may take various forms. Examples of different forms of a retaining ring are show in FIGS. 2-4. Other examples of retaining rings in accordance with this embodiment are possible as well.

FIG. 2A depicts a perspective view of a cartridge holder 1002 that has a retaining ring 1003 attached. In this embodiment, the retaining ring 1003 is attached at the proximal end 1000 of the cartridge holder 1002.

The ring 1003 may beneficially prevent a cartridge 1001 from being removed from the cartridge holder 1002. In other words, when the ring 1003 is attached to the holder 1002, the ring 1003 locks the cartridge 1001 within the holder 1002. FIG. 2B shows a cross-sectional perspective view of retaining ring 1003 attached at the proximal end 1000 of the cartridge holder 1002. As shown, a distal end 112 of the retaining ring 1003 abuts a proximal end 114 of the cartridge 1001. This ring 1003 does not allow the cartridge 1001 to move in the proximal direction 116. Although not shown, the cartridge 1001 is prevented from moving in a distal direction by the distal end of the holder 1002. Thus, the cartridge 1001 is securely retained in the holder 1002 and is prevented from moving in a distal and/or a proximal direction. Further, as shown in FIG. 2B there is a conical shape that may wedge the cartridge 1001 in place and, thus, prevent radial movement even if radial sizes vary due to tolerances.

The retaining ring 1003 may be connected to the holder 1002 in a variety of ways. For example, as shown in FIG. 2B, the ring 1003 may have a screwthread (e.g. threads 118, 120) that secures the retaining ring 1003 to the cartridge holder 1002 and can be tightened to remove axial play.

Further, in this embodiment depicted, the holder 1002 includes a fastening means 1004 for connecting to the dose setting mechanism 102 of the drug delivery device 100. The fastening means 1004 may include any fastening mechanism known in the art or later developed. As depicted, the fastening means 1004 includes a groove 124 that is capable of receiving and retaining a pin provided on a connecting feature of the dose setting mechanism 102. Beneficially, locating the fastening means 1004 on the holder 1002 may reduce tolerances between the device 100 and the datum face on the distal end of the cartridge 1001. In particular, the cartridge 1001 may sit on the datum face at the distal end of the holder 1002. The device 100 may then contact the circular datum face just to the distal side of fastening means 1004.

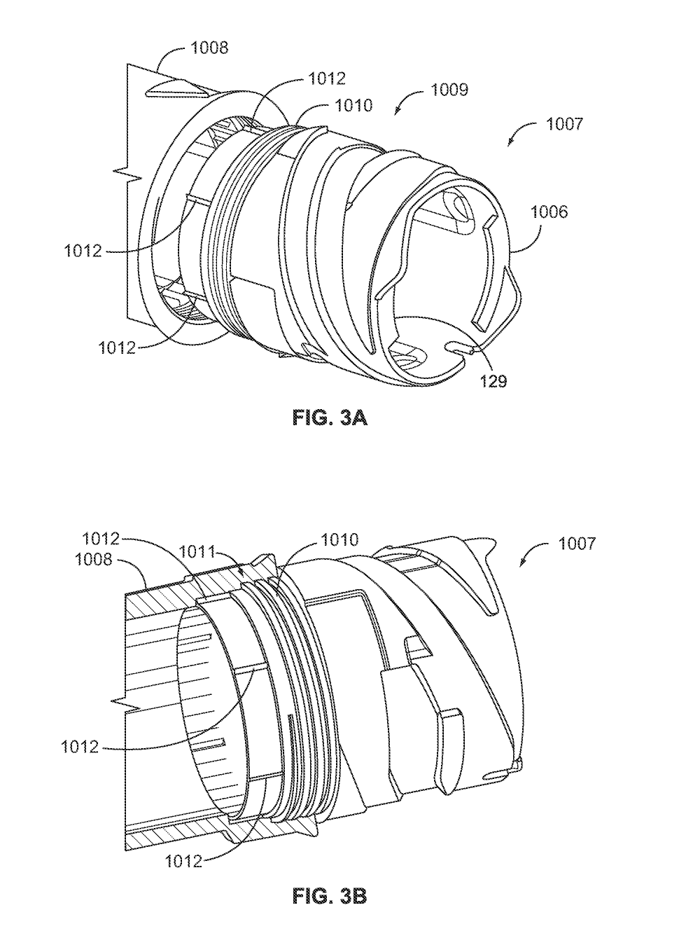

In another embodiment, the retaining ring itself may include the fastening means for connecting to the dose setting mechanism of the drug delivery device. Such an example of a retaining ring that includes a fastening means is depicted in FIGS. 3A-B. As seen in these Figures, retaining ring 1007 may be connected to cartridge holder 1008. Retaining ring 1007 includes fastening means 1009 for fastening to a drug delivery device. Additionally, the ring 1007 includes threads 1010 for connection to the holder 1008. So that the retaining ring 1007 acts to retain an inserted cartridge, the retaining ring 1007 may include at least one retention feature, such as retention features 1006, 129.

As shown in FIG. 3B, the holder 1008 has corresponding threads 1011 that are capable of receiving threads 1010, thus allowing the holder 1008 and ring 1007 to be securely attached. In certain embodiments, the retaining ring 1007 may have at least one ratchet feature, which may serve to beneficially prevent unscrewing of the thread 1011. A single ratchet feature may prevent unscrewing of the thread 1011. However, in certain arrangements, the retaining ring 1007 may include more than one ratchet feature. Multiple ratchet features may increase the difficulty of disengaging the ring 1007 from the holder 1008, as well as ensuring that the holder 1008 still has at least one ratchet feature should one or more be damaged. For example, retaining ring 1007 has a plurality of ratchet features 1012. These ratchet features 1012 may act to prevent a user from detaching the retaining ring 1007 after it has been initially attached to the holder 1008.

In addition to providing ratchet features 1012 shown in FIGS. 3A-B for preventing removal of the retaining ring 1007, other methods of preventing removal of the retaining ring 1007 are possible as well. For example, in certain embodiments, removal of the retaining ring 1007 may be prevented by glue and/or welding.

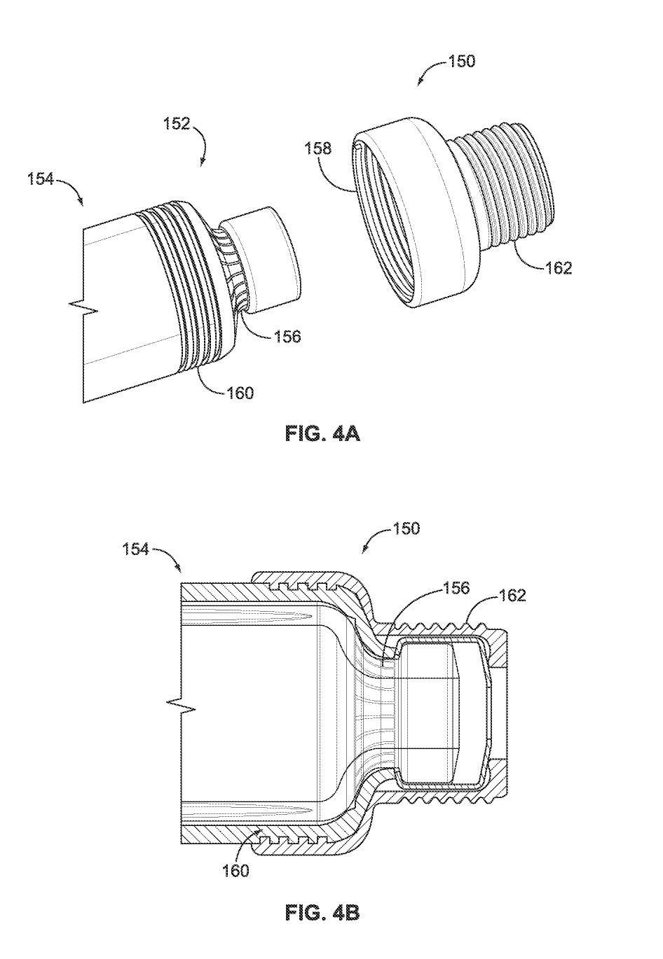

In yet another alternative arrangement, the interface between the cartridge holder and the retaining ring could be at the distal end of the cartridge holder. For example, the retaining ring could be located around the ferrule. An example of such an embodiment is depicted in FIGS. 4A-B. The holder 154 may include flexible protrusions 156 that act to retain or bias the cartridge. When the cartridge is inserted into the holder 154 in a distal direction, the flexible protrusions 156 expand radially outwards to pass over the ferrule of the cartridge and then, due to their elasticity, contract back inwards. FIG. 4A depicts a retaining ring 150 that may then connect to a distal end 152 of holder 154. This ring 150 may include threads 158 that allow the ring 150 to attach to threads 160 of the holder 154. When this ring 150 is secured in place, the flexible protrusions 156 cannot deflect, and the cartridge ferrule is gripped between the flexible protrusions and the distal end of the ring. The ring may further comprise threads 162 that allow the ring 150 to attach to a double-ended needle.

Beneficially, as mentioned above, a cartridge assembly can be coded to a given device (or given devices) so that the assembly only operates with (e.g. connects to) the given device (or given devices). The cartridge assembly (e.g. the cartridge holder and/or the retaining ring) may be coded to the given device in any way known in the art or later developed. For example, the assembly may be mechanically coded to the given device such that a mechanically coded assembly only connects to a correspondingly coded dose setting mechanism.

In a second embodiment for retaining a cartridge in a cartridge holder, a snap-on retaining ring may be used in order to retain the cartridge in the holder. This snap on retaining ring is similar in certain respects to the retaining ring discussed above with reference to FIGS. 2-4, and, thus, will not be described in as great of detail. However, it should be noted that many possibilities and permutations described above with respect to the first embodiment may equally apply to this second embodiment. For example, aside from including ratchet features to prevent removal of the retaining ring, all of the variations discussed with respect to the first embodiment are possible.

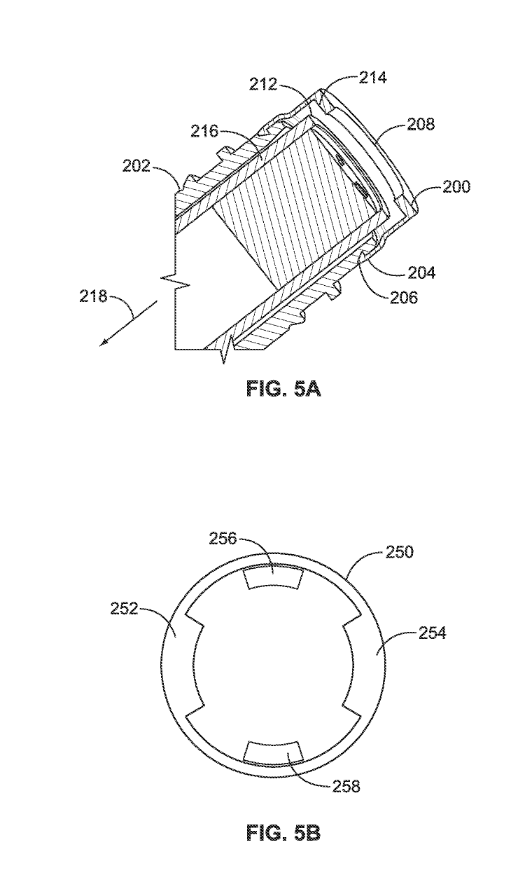

An example of the second embodiment where a snap-on retaining ring may be used to retain a cartridge in a holder is described with reference to FIGS. 5A-B. Snap-on retaining ring 200 may be attached to cartridge holder 202. The ring 200 may have a connection feature 204 and holder 202 may have a corresponding connection feature 206 that allow the two pieces to attach to one another (e.g. snap together). Snap-on ring 200 preferably includes at least one retention feature, such as retention features 208, 210. When the snap-on ring 200 is attached to the holder 202, these retention features 208, 210 may act to securely retain the cartridge 216 in the holder 202.

The snap-on retaining ring 200 preferably interfaces smoothly with the holder 202, so that the holder 202 and ring 200 may be attached to the dose setting mechanism 102. In an example of this second embodiment, the holder 202 may include the fastening means for connecting to the dose setting mechanism 102. In this example, the snap-on ring 200 is designed so as to not interfere with the fastening means. In yet another example, the retaining ring 200 itself may include the fastening means. In still yet another example, the retaining ring 200 and the holder 202 may each comprise a fastening means portion, and these portions may combine to create the fastening means that is necessary to connect to the dose setting mechanism 102.

The retaining features of this second embodiment that serve to retain the cartridge 216 to the holder 202 may vary. The retention feature 208 may need to extend inwards to the same diameter as the cartridge 216, so that it can prevent the cartridge 216 being removed. However, a cartridge biasing mechanism may also need to contact the cartridge 216 at this diameter. By reducing the angle of both the cartridge biasing system and the retention features 208, 210, both can be incorporated at the same diameter. For instance, the retention feature 208, 210 of the retaining ring 200 may extend over any angle up to 360 degrees. For example, the retention feature 208 (half of which is depicted in FIG. 5A) extends over a full 360 degrees. As another example, a retaining ring may have two (or more) retention features that each extend over X degrees, where X may be any number of degrees between 1 degree and 180 degrees. An example of a retaining ring having two features that extend over X degrees is shown in FIG. 5B. As seen in FIG. 5B, retaining ring 250 has retention features 252 and 254. The retention features 252 and 254 extend over approximately 45 degrees. Such retaining rings 250 may allow any biasing mechanism in the device 100 to contact the cartridge 216 directly. Further, as shown in FIG. 5B, protrusions 256, 258 are protrusions that are on the distal end of a biasing mechanism.

Alternatively, in this second embodiment, a spring may be added (e.g. molded in) between face 212 and face 214 to bias the cartridge 216 toward the distal end 218 of the cartridge 216. In an alternative arrangement, the snap-on retaining ring itself may be flexible, e.g., with locally thin walls, cut-outs, or a flexible material. A flexible retaining ring may act to appropriately bias a cartridge 216 as desired.

In a third embodiment for retaining a cartridge in a holder, a split ring may be used in order to retain the cartridge in the holder. Specifically, a split ring may be used to retain the cartridge behind the ferrule of the cartridge. Such a split ring may prevent movement of the cartridge by securely retaining the ferrule in the distal end of the holder.

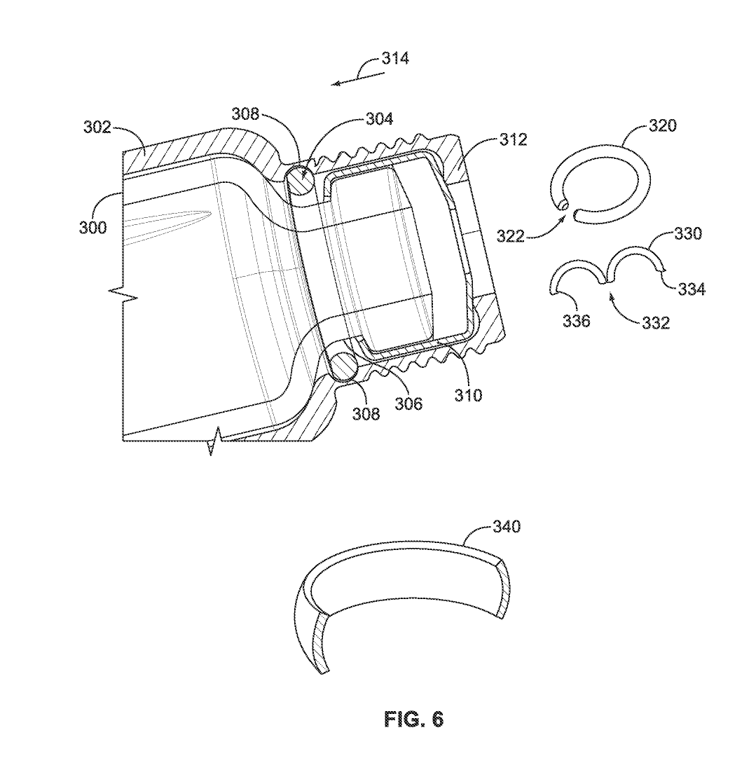

An example of the third embodiment is shown in FIG. 6, which depicts a perspective cross-sectional view of a distal end of a cartridge and holder. In this example, a cartridge 300 is placed in holder 302. A split ring 304 is placed over the neck 306 of the cartridge 300. When the holder 302 is pushed on or over the cartridge 300, the ring 304 compresses inwards, and then the ring 304 springs outward into a groove 308. When the ring 304 is secured in the groove 308, the ring 304 may act to retain the cartridge 300 in the holder 302. Preferably, the ring 304 is designed so that it has a larger diameter than the diameter of the groove 308 in the cartridge holder 302 for which it is designed. If the ring 304 has a larger diameter, the ring 304 may spring outwards to retain itself in the groove 308 when the cartridge 300 is fitted in the holder 302.

The ring 304 acts to retain the cartridge 300 because the ferrule 310 is held tightly between the ring 304 and a distal end 312 of the holder 302. The ring 304 prevents the cartridge 300 from moving in proximal direction 314. Further, the distal end 312 of the holder 302 prevents the cartridge 200 from moving in the distal direction. Thus, the cartridge 300 is securely retained in the holder 302.

A split ring in accordance with this embodiment may take a variety of forms, and examples of possible split rings are also depicted in FIG. 6. As a first example, split ring 320 is a generally circular ring with a small split 322. As another example, the split ring could be hinged and snap together. In this example, the ring may be relatively stiff when assembled, so the holder 302 may need to be flexible in order to assemble the cartridge 300. In this example, as the diameter of the ring is larger than the diameter of the neck of the cartridge holder 302, some deformation is needed when the cartridge 300 is fitted in the holder 302. The first ring 320 is flexible and, thus, can deform inwards. When assembled, the hinged ring forms a complete circle and is difficult to deform, so the holder 302 itself must deform. Such an example is depicted in FIG. 6. As shown, split ring 330 includes a hinge 332 between a first semi-circular portion and a second semi-circular portion of ring 330. The first semi-circular portion has a snap feature 334 that is complimentary to snap feature 336, which is on the end of the second semi-circular portion.

As yet another example, the split ring could be flexible in the axial direction. For example, split ring 340 may be flexible in the axial direction. In this example, if the split ring 340 sits between a distally facing shoulder of the cartridge holder 302 and a proximally facing shoulder of the ferrule 310, the split ring 340 can bias the cartridge 300 in the distal direction. The ring 340 may also include features so that the cartridge 300 is easy to insert but more difficult to remove, together with any modifications necessary to form these features by injection molding.

It should be understood that other types and variations of split rings are possible as well. Further, in addition to securing a cartridge in a holder, this embodiment may be used to retain a cap on a cartridge, where the assembled cartridge and cap can be inserted into a cartridge holder.

In this third embodiment, removal of the split ring could be prevented through the use of glue and/or welding. Further, as discussed above with respect to the first and second embodiments, a cartridge assembly in accordance with this third embodiment can be coded to a given device (or given devices) so that the assembly only operates with (e.g., connects to) the given device (or given devices).

In a fourth embodiment for retaining a cartridge in a cartridge holder, a gap filling material such as an adhesive and/or resin may be used in order to retain the cartridge in the holder. Suitable materials might be polypropylene, polyurethane, polyester, or epoxy. The adhesive and/or resin may be applied in various places and in various ways in order to retain the cartridge. In an example, the adhesive and/or resin may be applied along a cartridge sidewall. As another example, adhesive and/or resin may be injected into a void between the cartridge neck and the cartridge holder. This adhesive and/or resin could then be cured in order to retain the cartridge in the holder.

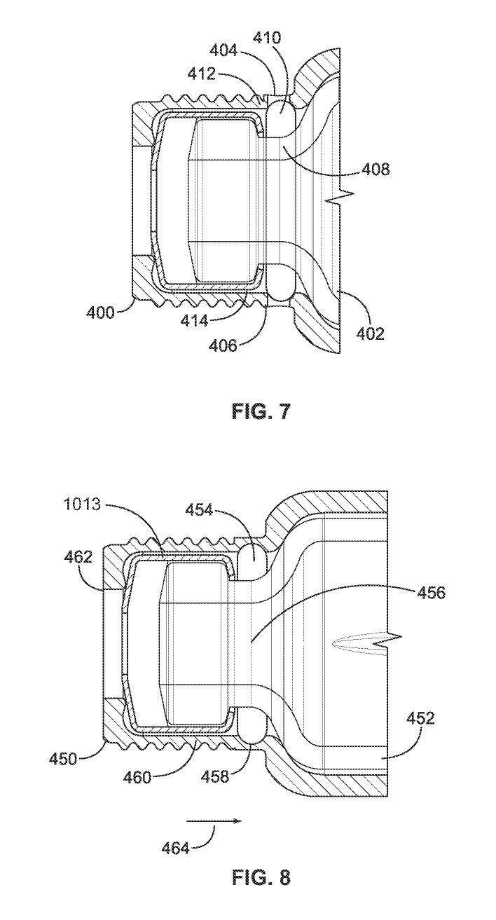

Examples of this fourth embodiment are shown in FIGS. 7-8. FIG. 7 depicts a distal end of cartridge holder 400 that has a cartridge 402 inserted. Cartridge holder 400 may have an injection port or ports, such as injection ports 404, 406 located at or near where the neck 408 of cartridge 402 resides when cartridge 402 is fully inserted in cartridge holder 400. Adhesive 410 may be injected into injection port 404 and/or 406. The adhesive 410 may fill in the void between the cartridge holder sidewalls 412, 414 and the neck 408 of the cartridge 402. As mentioned above, this adhesive 410 may be cured in order to securely retain the cartridge 402 in the holder 400.

As another example of using adhesive or resin to retain a cartridge in a holder, one may refer to FIG. 8. In this example, the adhesive may extend into a groove in the holder. The adhesive or resin may not bond well to a smooth surface, so the groove may include features such as axial grooves/ridges to improve ‘keying’. Since the adhesive must shear to release the cartridge, a material with a weaker adhesive bond may be used, increasing the choice of suitable materials. FIG. 8 depicts a distal end of cartridge holder 450 that has cartridge 452 fully inserted. As shown in FIG. 8, adhesive 454 is filled in around the neck 456 of the cartridge 452. This adhesive 454 fills in groove 458 in the sidewall 460 of the cartridge holder 450. The adhesive 454 beneficially acts to retain the cartridge 452, as the ferrule 1013 is held tightly between the adhesive 454 and a distal end 462 of the holder 450. The adhesive 454 prevents the cartridge 452 from moving in proximal direction 464, and the distal end 462 prevents the cartridge 452 from moving in the distal direction. Therefore, the cartridge 452 is securely retained in the holder 450 and cannot be removed.

In certain examples of this fourth embodiment, before fixing the cartridge 452 in the one-piece cartridge holder 450 with adhesive or resin 454, the cartridge 452 may be pushed axially to bias the cartridge 452 against the datum at the distal end of the cartridge holder 450 (i.e. the end where the double needle is attached).

Further, in certain examples of this fourth embodiment, a detachable membrane placed over the ferrule 1013 may prevent adhesive 454 from leaking on to the septum. Still further, a visual inspection (e.g. by quality control personnel or equipment in a manufacturing line) may ensure that no adhesive 454 has reached onto the septum.

It should be noted that the same or a similar method in accordance with this fourth embodiment may be used to secure a cap onto a cartridge.

Further, as discussed above with respect to the first through third embodiments, a cartridge assembly in accordance with this fourth embodiment may beneficially be coded to a device. For example, the holder may be coded to the device in the ways discussed above.

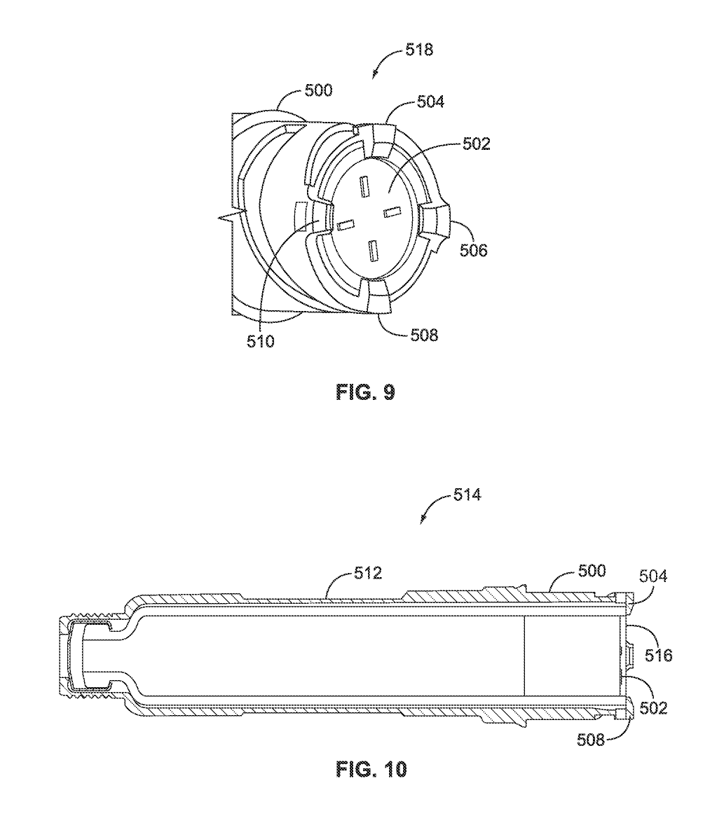

In a fifth embodiment for retaining a cartridge in a cartridge holder, a one-piece holder with retention features may be used in order to retain the cartridge in the holder. In this fifth embodiment, the cartridge holder may be a single piece, and this single-piece holder may have at least one retention feature disposed on the proximal end that acts on the proximal end (i.e. the open end) of cartridge that is inserted in the holder. Examples of the fifth embodiment are shown in FIGS. 9, 10, 11A-B.

FIG. 9 depicts a perspective view of a proximal end of cartridge holder 500 having cartridge 502 fully inserted. Cartridge holder 500 has retention features 504, 506, 508, 510 disposed on the proximal end. These retention features 504, 506, 508, 510 retain cartridge 502 in holder 500, as the cartridge 502 cannot be removed from the holder 500 due to retention features 504, 506, 508, 510. In addition, the one-piece holder 500 includes fastening mechanism 518 that may allow the holder 500 to attach to a dose setting mechanism. Although these retention features 504, 506, 508, 510 shown in FIG. 9 are depicted as tabs, it should be understood that the retention feature may be a ring that covers the full circumference of the proximal end of the holder 500.

In an example of this embodiment, the cartridge holder 500 may deform axially during cartridge insertion, which may ensure that the retention feature(s) 504, 506, 508, 510 of the holder 500 is pressed against the proximal end of the cartridge 502. For instance, a flexible section of the holder 500 may stretch to accommodate the length of the cartridge 502. Such a flexible section may be formed by locally thin walls, cut-outs, or a flexible material, such as polypropylene (PP), polyamide (PA) or acetal (POM). The flexible section may be located in various positions. For example, the flexible section may be in an intermediate position (e.g. a center position) of the holder. FIG. 10 depicts a cross-sectional view of cartridge holder 500 and cartridge 502. Cartridge holder 500 includes a flexible section 512 in a center section 514 of the holder 500. When the cartridge 502 is inserted into the holder 500, the cartridge 502 may force the flexible section 512 to expand. This expansion acts to ensure that the retention features 504, 506, 508, 510 are pressed against the proximal end 516 of the cartridge 502, and hence that the cartridge 502 is biased in a distal direction relative to the delivery device 100.

In an alternative arrangement, the flexible portion may be located between the fastening means of the holder 500 and the retaining means. This location may beneficially reduce tolerances between the device 100 and the datum face on the distal end of the cartridge 502.

In certain arrangements, in order to ensure that the cartridge 502 is biased in a distal direction, a spring (not depicted) may be inserted between the cartridge 502 and retention feature(s) 504, 506, 508, 510. For example, with reference to FIG. 9, springs could be inserted between cartridge 502 and retention features 504-510. As will be appreciated by one of skill in the art, the example in FIGS. 9 and 10 may require complex tooling to mold as one part.

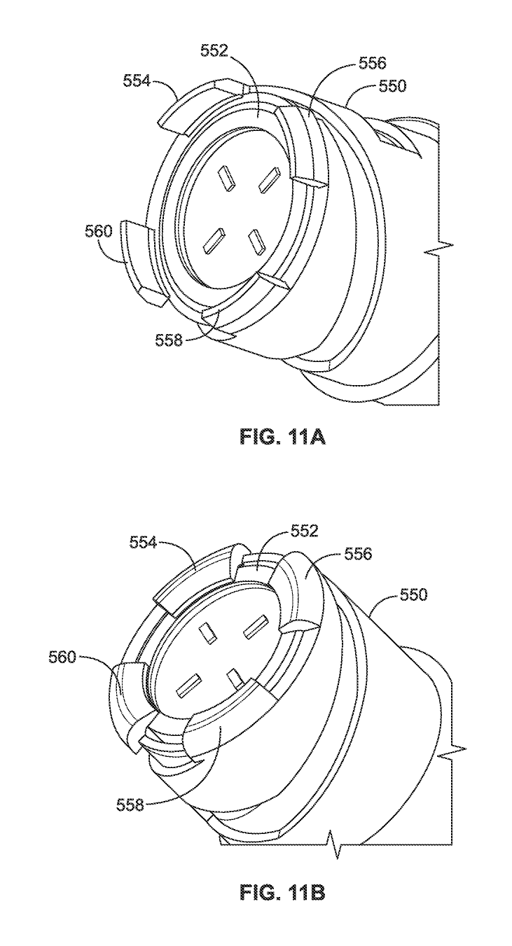

FIGS. 11A and 11B depict an alternative example of a one-piece holder with retention features that may be used in order to retain a cartridge in a holder, and may be easier to mold. Specifically, these Figures depict a proximal end of cartridge holder 550 having cartridge 552 fully inserted. Cartridge holder 550 comprises a plurality of flexible retention features 554, 556, 558, 560. These retention features 554, 556, 558, 560 are folding tabs. Before the cartridge 552 is inserted into the holder 550, these tabs are arranged in an upright position, as shown in FIG. 11A. After cartridge 552 is inserted into the holder 500, these folding tabs may be folded down. The tabs may snap down and thereafter serve to retain the cartridge 552, as shown in FIG. 11B. In this example, these retention features 554, 556, 558, 560 are retained by snap-fit features (not shown). However, retention features 554, 556, 558, 560 may be retained in other ways. For example, residual forces, heat, adhesive, and/or welding may be used to retain the retention features 554, 556, 558, 560. In given embodiments, once the tabs are pushed down, the tabs may be permanently fixed in a down position, and therefore are unable to return to an upright position. Therefore, in such an embodiment, the cartridge 552 would be non-detachably retained.

As mentioned above with respect to FIG. 9, 10, 11A, and 11B, the retention features may be located on the proximal end of the holder 500, 550. Additionally or alternatively, retention features may be located at the distal end of the one-piece holder 500, 550 in order to prevent movement of the ferrule. As yet another alternative, the one-piece cartridge holder 500, 550 in accordance with this fifth embodiment could contract in the radial direction and, hence, retain the cartridge 502, 552 by friction. For example, the holder 500, 550 may have a flexible cross-sectional shape. As another example, the holder 500, 550 could have axial splines. As yet another example, the holder 500, 550 (or part of the holder) could be made from a heat-shrink material that shrinks around the cartridge upon assembly.

The retention features in accordance with this fifth embodiment may be formed in various ways. For example, the retention features may be molded or formed by application by heat after the cartridge 502, 552 is inserted into the holder 500, 550.

Further, as discussed above with respect to the first through fourth embodiments, a cartridge assembly in accordance with this fifth embodiment may beneficially be coded to a device 100. For example, the holder 500, 550 may be coded to the device 100 in the ways discussed above.

In a sixth embodiment for retaining a cartridge in a holder, a holder that is connected with a snap-fit may be used in order to retain the cartridge in the holder. The holder connected with a snap-fit may retain the cartridge either at the distal end of the cartridge around the ferrule or at the proximal end of the cartridge. Similar to the embodiments discussed above, the snap-fit holder may have retention features that act to retain the cartridge. Alternatively, the snap-fit holder may provide a radial force over the cartridge that retains the cartridge by the force of friction. In other words, when the holder is snapped together, the holder may squeeze the cartridge and prevent movement in a proximal, distal, or radial direction.

In this embodiment, a holder may be split axially, and the holder may be clipped together after a cartridge is inserted. The clipped holder may act to securably retain the cartridge. The holder may be a single piece having a hinge. Alternatively, the holder may be more than one piece that is designed to connect to the other pieces.

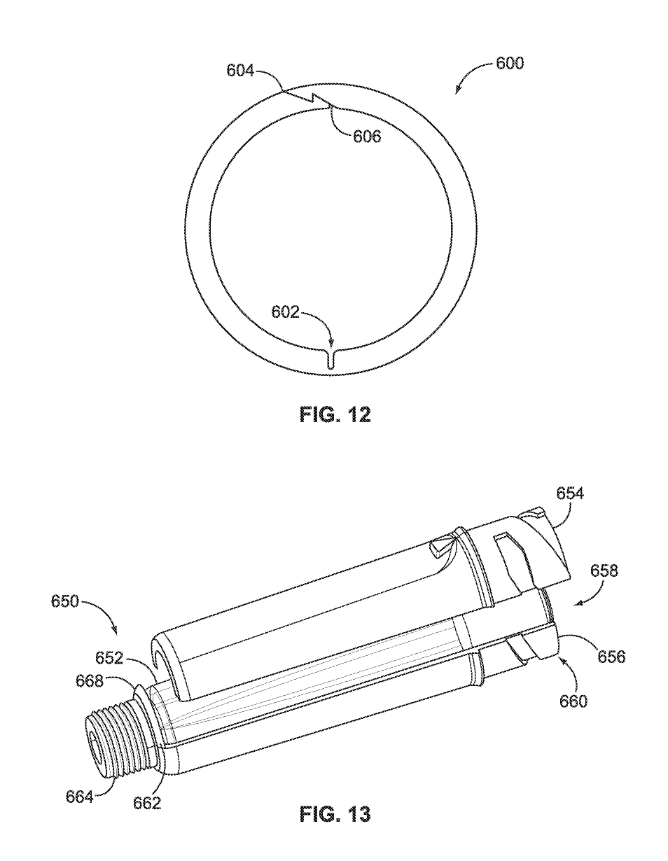

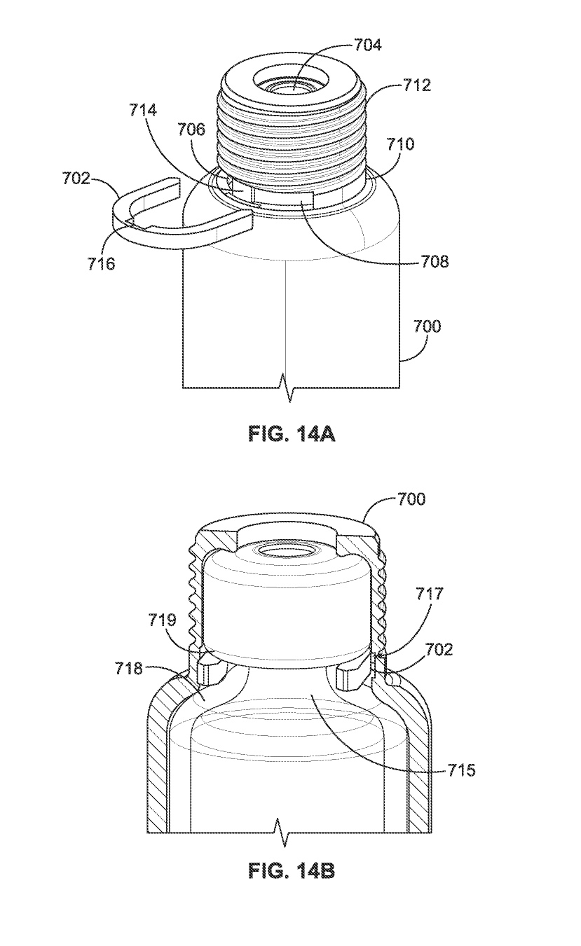

FIG. 12 depicts a cross section of a holder 600 that is a one piece holder having a hinge 602. The holder 600 has snap-fit features 604, 606 that snap together when pressed together. FIG. 13 depicts a perspective view of a two-piece holder 650 for retaining cartridge 652. Specifically, two-piece holder 650 contains a first piece 654 and a second piece 656. In this example, the split line 658 runs from the proximal end 660 of the cartridge 652 to point 662. Point 662 is located at the proximal end 668 of the needle thread 664. Since the split line in this example does not go through the needle thread, this beneficially reduces difficulties in rotating a double ended needle over the needle thread.

In another example, the split line could extend along the entire central longitudinal plane of the holder 650. In this example, the split line would split the needle thread (e.g. similar to the needle thread 664) in half.

In accordance with examples of this sixth embodiment, a molded feature or spring may be added to the snap-fit cartridge holder 650 in order to bias the cartridge 652 toward the distal end of the holder 650.

In this sixth embodiment, detachment of the two pieces of the holder 650 may be prevented through the use of glue and/or welding. Further, as discussed above with respect to the first and fifth embodiments, a cartridge assembly in accordance with this sixth embodiment can be coded to a given device 100 (or given devices) so that the assembly only operates with (e.g. connects to) the given device 100 (or given devices).

In a seventh embodiment for retaining a cartridge in a holder, an external clip may be used in order to retain a cartridge in a holder. After a cartridge is inserted into a cartridge holder, a clip applied from the outside of the holder may retain the holder around the ferrule. For example, the clip may be a u-beam that is inserted into a cut-out in a cartridge holder. This u-beam may be permanently retained with snap-fit features into the holder, or around the cartridge neck. Additionally or alternatively, the u-beam may be retained (i.e. fixed in place) with adhesive or resin. Preferably, after the clip is inserted into the holder, the clip is non-detachable. FIGS. 14-18 depict examples of this seventh embodiment for retaining a cartridge in a holder. It should be understood, however, that in addition to these depicted examples other variations are possible as well.

In this seventh embodiment, as discussed above with respect to the first and sixth embodiments, a cartridge assembly in accordance with this seventh embodiment can be coded to a given device (or given devices) so that the assembly only operates with (e.g. connects to) the given device (or given devices).

An exemplary cartridge holder 700 that uses an external clip 702 to retain cartridge 704 is shown in FIGS. 14A-B. The external clip is a u-beam (i.e. u-shaped clip). The clip 702 may be inserted around the neck of cartridge 704. In order to facilitate this insertion, cartridge holder 700 may have a hole or holes in the neck 710, such as holes 706, 708. As depicted, these holes 706, 708 are located beneath the threads 712, where a double ended needle may attach to the cartridge holder 700. A stop feature 714 may act as a stop to stop the clip 702 from moving beyond the feature 714. This stop feature 714 may also serve to strengthen the holder 700, which may beneficially prevent this top portion of the holder 700 from damage (e.g. breaking) The clip 702 may further have an indentation 716 that corresponds to the stop feature 714, allowing the clip 702 to be inserted so that the outside of the clip 702 is flush or substantially flush with the surface of the holder 700 when inserted. Further, this stop feature 714 and indentation 716 may act together to snap into one another, so as to secure the clip 702 to the holder 700.

FIG. 14B depicts a perspective view of the cartridge 704 and a perspective cross-sectional view of the holder 700. As can be seen, the clip 702 fits tightly around the neck 715 of the cartridge 704. The clip 702 contacts the bottom side 719 of the ferrule, and is prevented from axial movement relative to the cartridge holder 700 for example by contact around the holes 706 and 708 and grooves 717. This contact securely retains the cartridge 704 in place. As can be seen, the ferrule is retained because it cannot move past the clip 702. Preferably, the clip 702 does not contact the shoulder 718 of the cartridge 704. The distal end of the cartridge 704 preferably contacts the end of the holder 700.

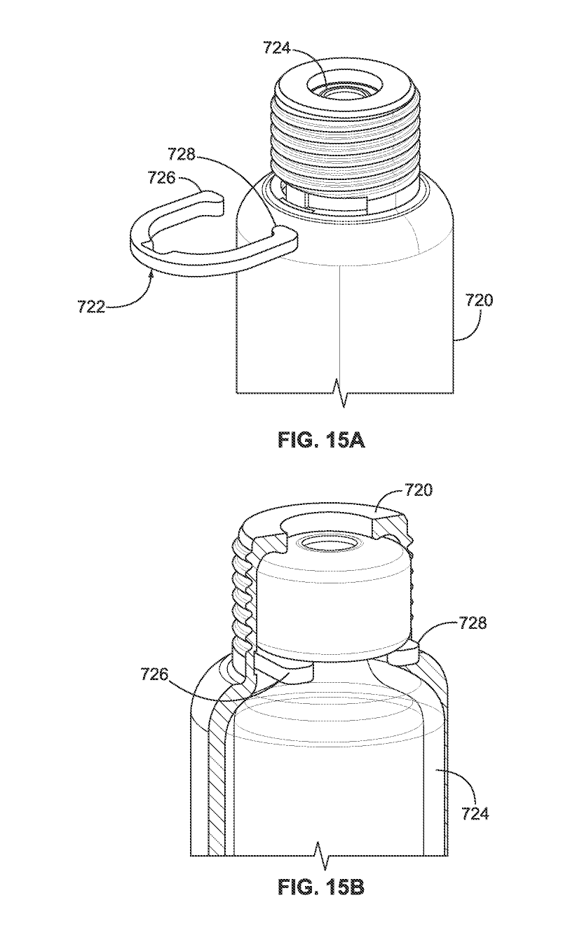

In another arrangement, the clip could be retained with snap-fit features around the neck of the cartridge. FIG. 15A depicts a cartridge holder 720 that uses an external clip 722 to retain cartridge 724. FIG. 15B depicts a perspective view of the cartridge 724 and a perspective cross-sectional view of the cartridge holder 720. This clip 722 and cartridge holder 720 is similar in many respects to cartridge holder 700 and clip 702 and, thus, will not be described in as great of detail. However, clip 722 is a slightly modified version of clip 702. As can be seen, clip 722 includes snap-fit features 726, 728 located at the end of each arm of the clip 722. As seen in 15B, these snap-fit features 726, 728 wrap around the neck of the cartridge 724. These features 726, 728 may provide additional retention forces that prevent the cartridge 724 from moving (see FIG. 15B).

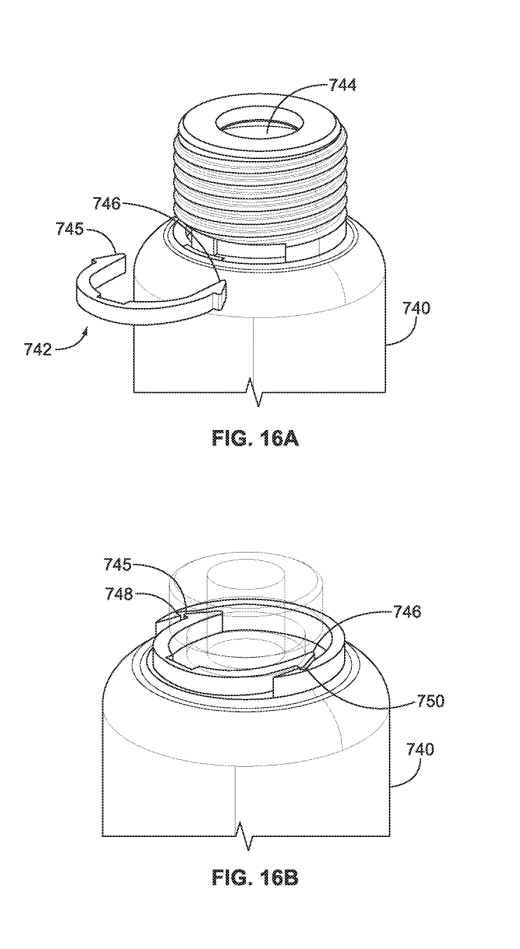

In yet other arrangements, the clip could be retained with snap-fit features that snap into the holder. FIG. 16A depicts a cartridge holder 740 that uses an external clip 742 to retain cartridge 744. FIG. 16B depicts a perspective view of the cartridge 744 and a perspective cross-sectional view of the cartridge holder 740. This clip 742 and cartridge holder 740 is similar in many respects to the clips and cartridge holders described above and, thus, will not be described in as great of detail. However, the clip 742 and holder 740 are slightly modified versions of the clip and holder described above. Clip 742 includes protrusions 745 and 746 and holder 740 includes indentations 748 and 750. When the clip 742 is inserted into the holder 740, the protrusion 745 interacts with the indentation 748 and protrusion 746 interacts with indentation 750. That is, the protrusions 745. 746 snap into the indentations 748, 750, and this interaction retains the external clip 742 securely to the holder 740 (see FIG. 16B).

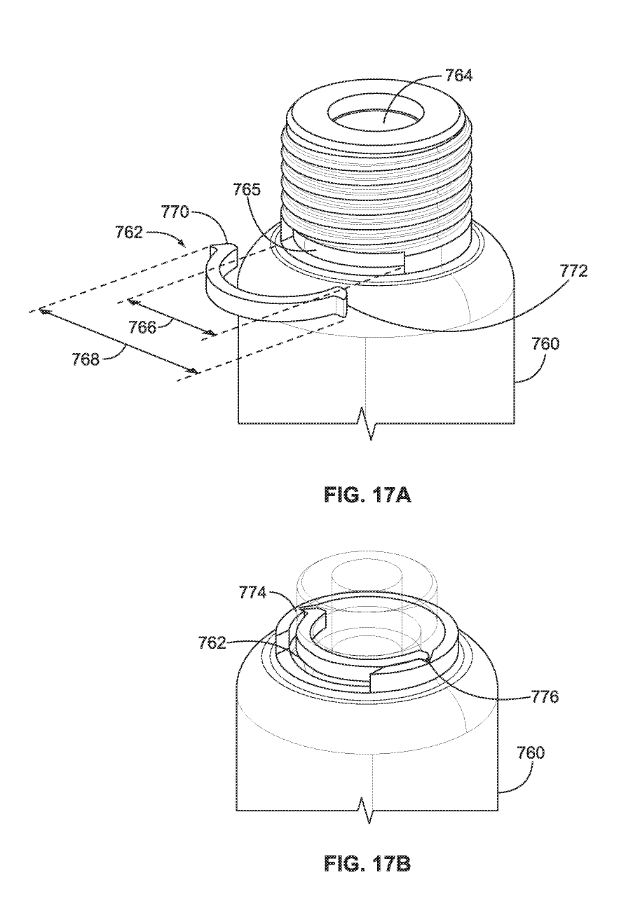

FIG. 17A depicts a cartridge holder 760 that uses an external clip 762 to retain cartridge 764. FIG. 17B depicts a perspective view of the cartridge 764 and a perspective cross-sectional view of the cartridge holder 760. This clip 762 and cartridge holder 760 is similar in many respects to clips and cartridge holders described above and, thus, will not be described in as great of detail. However, this holder 760 does not include a stop feature that serves to stop the clip 762 when it is inserted into the holder 760. Rather, the hole 765 and the clip 762 are designed such that the hole 765 is more narrow than the clip 762. As shown, the hole 765 has a length 766 and the clip 762 (in expanded form) is a length 768 that is greater than length 766. When the clip 762 is attached, the clip 762 may be compressed to fit through the hole 765, and then the clip 762 may expand in order to retain itself Any resilient material may be used for the clip 762, for example metal such as steel, or polymers such as PP, PA or POM. Further, in this example, clip 762 has protrusions 770, 772 that fit into indentations 774, 776 on the inner wall of the holder 760, and these may interact to securely connect the clip 762 to the holder 760. Still further, there may be a transverse face (not shown) at the bottom of indentations 774, 776 that prevent axial movement of the clip 762.

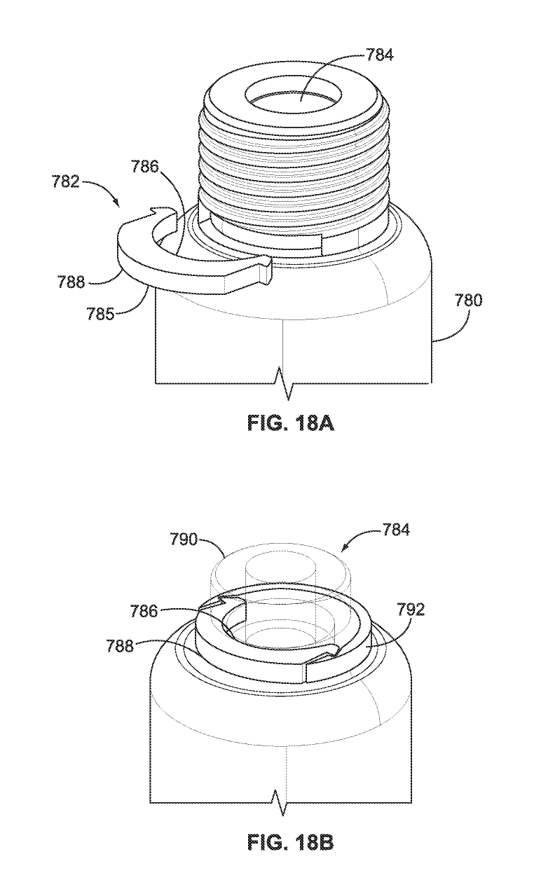

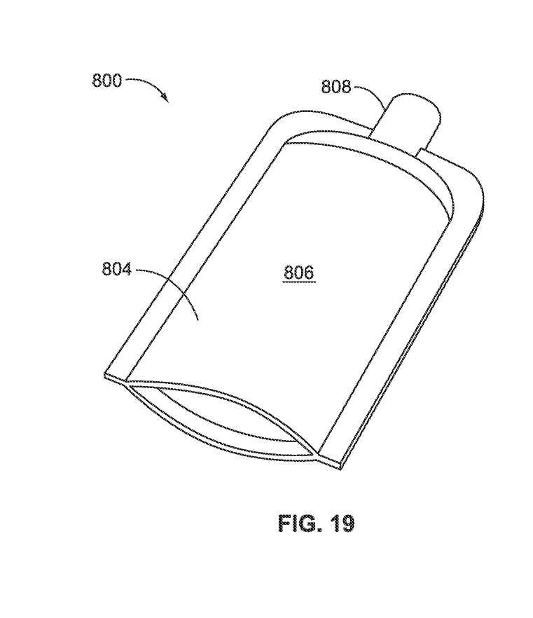

FIG. 18A depicts a cartridge holder 780 that uses an external clip 782 to retain cartridge 784. FIG. 18B depicts a perspective view of the cartridge 784 and a perspective cross-sectional view of the cartridge holder 780. This clip 782 and cartridge holder 780 is similar in many respects to clips and cartridge holders described above and, thus, will not be described in as great of detail. In this example, when the clip 782 is fully inserted, clip 782 is flush with the outside of the holder 780. Beneficially, being flush with the outside of the holder 780 may make the clip 782 more difficult to remove. The body 785 of the clip 782 is designed so that a first side 786 is flush with ferrule 790 and a second side 788 is flush with the outer diameter 792 of the holder 780. However, it should be understood that in certain embodiments, the clip 782 is not necessarily flush with the outside of the holder 780.

In addition to the examples depicted in FIG. 14-18, other external clip arrangements are possible as well. For example, multiple external clips may be used to retain the cartridge.

Although aimed primarily at the insulin market, the disclosed concepts may apply to other drugs. The disclosure may apply to various devices, including the following examples:

An injector pen with a cartridge (e.g. 3 ml cylindrical glass cartridge) non removably retained in a holder, so that the holder will be disposed of with the primary pack.

Any drug delivery device with any type of primary pack, e.g. inhaler, pouch.

Although described primarily with reference to a cartridge assembly that attaches to an injection pen-type drug delivery device, the disclosed embodiments may apply to any drug delivery device, with any type of reservoir or primary pack, e.g. inhaler, pouch. For example, FIG. 19 illustrates a drug reservoir 800 comprising a vessel 804 that contains a medicament 806. A stopper 808 is provided along a distal end of the vessel 804 and is attached to the vessel 804 so as to prevent the medicament 806 from exiting the vessel 804. Such a pouch may be retained in an appropriate pouch holder using the embodiments discussed above for retaining a cartridge in a holder.

The disclosed embodiments for retaining a cartridge in a holder result in a number of advantages. For example, a holder that contains a non-detachable cartridge can be coded (e.g. mechanically coded) to a specific device, ensuring that a drug can only be used with the correct device. Further, assembly of the cartridge assembly is relatively easy and disassembly of the cartridge assembly is difficult. As mentioned, in certain embodiments, the cartridge is non-detachably retained in the holder. Still further, in given embodiments, no biasing mechanism is needed within the drug delivery device, which may conserve materials and/or manufacturing costs, and provide a more robust solution than a biasing mechanism within the device. Further, the disclosed embodiments may beneficially limit tampering with or counterfeiting of cartridge assemblies. The disclosed concepts may provide a dedicated cartridge assembly that is difficult to tamper with so that the cartridge may not be compromised in that the cartridge can be used with an unauthorized drug or drug delivery device. Because such cartridges may be difficult to tamper with, they may also reduce the risk of counterfeiting: i.e. making it more difficult for counterfeiters to provide unregulated counterfeit medicament carrying products.

Exemplary embodiments of the present invention have been described. However, as those of skill in the art will recognize certain changes or modifications to such arrangements may be made. Those skilled in the art will understand, however, that further changes, modifications, revisions and/or additions may be made to the presently disclosed arrangements without departing from the true scope and spirit of the present invention, which is defined by the claims.

1-20. (canceled)

21. A cartridge assembly for connection to a drug delivery device, the cartridge assembly comprising:
a cartridge holder;
a drug cartridge held in the cartridge holder;
a retaining member;
wherein the retaining member is connected to the cartridge holder so that the retaining member retains the drug cartridge in the cartridge holder,
and wherein the retaining member is a retaining ring.

22. The cartridge assembly of claim 21, wherein the retaining member is connected to a proximal end of the cartridge holder.

23. The cartridge assembly according to claim 21, wherein at least one of the retaining member and the cartridge holder is mechanically coded to a given drug delivery device.

24. The cartridge assembly according to claim 21, wherein the retaining ring comprises at least one connection feature for connecting the retaining ring to the cartridge holder, and wherein the at least one connection feature is a threaded connection feature or at least one protrusion, wherein the protrusion is capable of snapping into an indentation in the proximal end of the cartridge holder.

25. The cartridge assembly according to claim 21, wherein the retaining ring comprises at least one ratchet feature located on an outer wall of the retaining ring.

26. The cartridge assembly according to claim 21, wherein the retaining ring comprises at least one retention feature extending from an inner wall of the retaining ring, wherein the at least one retention feature is a protrusion that protrudes along an entire circumference of the inner wall of the cartridge holder, or wherein the at least one retention feature comprises a plurality of tabs that extend from the inner wall of the retaining ring.

27. The cartridge assembly according to claim 21, wherein the retaining ring is connected to a distal end of the cartridge holder, and wherein the retention feature retains a ferrule of the drug cartridge.

28. The cartridge assembly according to claim 21, wherein the retaining ring is flexible such that the ring biases the drug cartridge toward a distal end of the cartridge holder.

29. The cartridge assembly according to claim 21, wherein the retaining ring comprises a fastening mechanism for attaching the cartridge holder to the drug delivery device, wherein the fastening mechanism is located on an outer wall of the retaining ring.

30. A cartridge assembly for connection to a drug delivery device, the cartridge assembly comprising:
a cartridge holder;
a drug cartridge held in the cartridge holder;
a retaining member;
wherein the retaining member is connected to the cartridge holder so that the retaining member retains the drug cartridge in the cartridge holder.

31. The cartridge assembly of claim 30, wherein the retaining member is connected to a proximal end of the cartridge holder.

32. The cartridge assembly according to claim 30, wherein at least one of the retaining member and the cartridge holder is mechanically coded to a given drug delivery device (100).

33. The cartridge assembly according to claim 30, wherein the retaining member is a split ring, the split ring being disposed between a portion of the cartridge holder and a portion of the drug cartridge.

34. The cartridge assembly according to claim 33, wherein at least one of the split ring and the cartridge holder is flexible, and wherein an angle of at least one contact face between the split ring and the cartridge holder allows for assembly of the cartridge assembly and prevents disassembly of the cartridge assembly.

35. The cartridge assembly according to claim 30, wherein the retaining member is a clip, and wherein a neck portion of the cartridge holder comprises at least one hole for receiving the clip, wherein the clip is inserted into the hole.

36. The cartridge assembly according to claim 35, wherein the cartridge holder comprises connection features on an inner wall of the neck portion of the cartridge holder, and wherein the clip comprises corresponding connection features that fit into the connection features of the holder.

37. The cartridge assembly according to claim 30, wherein the retaining member comprises at least one of an adhesive and a resin disposed between a portion of the cartridge holder and a portion of the drug cartridge, wherein the at least one of an adhesive and a resin retains the cartridge in the cartridge holder.

38. The cartridge assembly according to claim 37, wherein a neck of the cartridge holder comprises at least one injection port through which the adhesive may be injected.

39. The cartridge assembly according to claim 37, wherein the drug cartridge includes a ferrule, wherein the ferrule has a detachable membrane provided over the ferrule, and wherein the detachable membrane is configured to prevent adhesive from touching a septum in the ferrule.

40. A cartridge assembly for connection to a drug delivery device, the cartridge assembly comprising:
a cartridge holder
a drug cartridge held in the cartridge holder,
wherein the cartridge holder comprises at least one fastening feature for connecting to a drug delivery device and at least one retention feature, wherein, when the drug cartridge is held in the cartridge holder, the retention feature prevents the drug cartridge from moving past a proximal end of the cartridge holder, wherein the at least one retention feature is located on a proximal end of the cartridge holder, or wherein the at least one retention feature is located at a distal end of the cartridge holder, and wherein the retention feature prevents movement of the ferrule of the drug cartridge.
